(19) United States
(12) Patent Application Publication
(10) Pub. No.: US 2010/0298677 A1
(43) Pub. Date: Nov. 25, 2010

(54) WIRELESS RING-TYPE PHYSICAL DETECTOR

(75) Inventors: CHUN-HAO LU, TAINAN COUNTY (TW); CHENG-CHI TAI, TAINAN COUNTY (TW); JIUN-HUNG LIN, TAINAN COUNTY (TW)

Correspondence Address: ROSENBERG, KLEIN & LEE, 3458 ELLICOTT CENTER DRIVE-SUITE 101, ELLICOTT CITY, MD, 21043, US

(73) Assignee: ASTEK TECHNOLOGY LTD., TAINAN COUNTY (TW)

(21) Appl. No.: 12/470,868

(22) Filed: May 22, 2009

(51) Int. Cl. A61B 5/02; A61B 5/1455

(52) U.S. Cl. 600/324

(57) ABSTRACT

A wireless ring-type physical detector includes a ring, a sensor unit, an amplifier unit, a demultiplexer unit, a processor unit and a wireless transmission unit. The sensor unit uses a light signal to detect the blood oxygen saturation, the heartbeat and continuous blood pressure. The detected light signal is processed by each unit to get a physical parameter which is valuable for a clinic test.

### BACKGROUND OF THE INVENTION

1. Field of the Invention

The present invention relates to a wireless ring-type physical detector, and more particularly to a light sensor to detect the blood oxygen saturation, the heartbeat and the continuous blood pressure which are valuable for a clinic test.

2. Description of the Prior Art

There are three types of hemadynamometer used to get blood measurement on the market. These are mercury column, air pressure, and electronic types. The mercury column type has a better accuracy and reliability. But, it is not made for mobility and requires using a stethoscope to work with. The air pressure type is also called a liquidless tonometer, such as a clock that uses a mechanical movement to present blood readings. This is easy to carry and simple in operation. However, the accuracy is not as good as the mercury column type and it is complicated for maintenance. The electronic type is light weight and easy to carry and to operate. However, the accuracy is also a big concern.

In order to solve the problems as mentioned above, an invention is derived, Taiwanese Patent Publication No. M290417 titled “ring measuring device”, which comprises a case to accommodate a sensor and a microcontroller. The case is in a ring type for the user to sleeve on his/her finger with the sensor to detect a signal from the finger and send the signal to the microcontroller for processing to come up with a readable result.

However, the result came from the heartbeat and the blood oxygen saturation is not stable, which may vary depending upon the person's situation.

### SUMMARY OF THE INVENTION

Due to the shortcomings of the prior art, the present invention provides a wireless ring-type physical detector which comprises a light sensor to detect the blood oxygen saturation, the heartbeat and the continuous blood pressure for providing more accurate information.

According to the present invention, there is provided a wireless ring-type physical detector, comprising:

a ring, the ring comprising a chamber, an inner surface, and a display screen;

a sensor unit, the sensor unit comprising two transmitters and a receiver disposed on the inner surface of the ring, the transmitters and the receiver using a light signal to detect the blood oxygen saturation, the heartbeat and continuous blood pressure and transforming the light signal into a current signal;

an amplifier unit, the amplifier unit being disposed in the chamber of the ring and connected to the sensor unit for transforming the current signal into a voltage signal;

a demultiplexer unit, the demultiplexer unit being disposed in the chamber of the ring and connected to the amplifier unit for outputting a demultiplex signal after selection and allotment;

a processor unit, the processor unit being disposed in the chamber of the ring and connected with the sensor unit, the amplifier unit, and the demultiplexer unit for controlling each unit, the processor unit receiving and calculating the demultiplex signal to come up with a physical parameter and outputting the physical parameter to the display screen; and

a wireless transmission unit, the wireless transmission unit being disposed in the chamber of the ring and connected to the processor unit, the wireless transmission unit receiving and transmitting the physical parameter.

Preferably, the ring comprises a power supply unit therein.

Preferably, the two transmitters of the sensor unit are disposed on a first side of the inner surface of the ring, the light signal from the transmitters pointing at a first position at a second side of the inner surface of the ring, the receiver being located at the first position.

Alternatively, the two transmitters of the sensor unit are disposed on a first side of the inner surface of the ring, the light signal from the transmitters pointing at a second side of the inner surface of the ring and returning back to a second position at the first side of the inner surface, the receiver being located at the second position.

Preferably, the amplifier unit is a transimpedence amplifier.

Preferably, the processor unit comprises an analog digital transformer for transforming a signal to be transmitted to the processor unit into a digital signal.

Preferably, the processor unit comprises a digital analog transformer for transforming an output signal into an analog signal before transmission.

Preferably, the wireless transmission unit is a wireless transmission module selected from one of infrared ray, Bluetooth, 5 KHz, 2.4 GHz, and Zigbee.

Preferably, the physical parameter is one of the blood oxygen saturation, the heartbeat and the continuous blood pressure.

Preferably, the ring further comprises a push button, the push button being connected with the processor unit for selecting an operating mode.

Preferably, the ring further comprises an adjusting belt for changing an inner diameter of the ring.

Preferably, the wireless ring-type physical detector further comprises a pedometer unit, the pedometer unit being disposed in the chamber of the ring and connected to the processor unit for sensing the step vibration of a user.

It is the primary objective of the present invention to provide a wireless ring-type physical detector, which provides a more accurate reading of a blood pressure.

It is another objective of the present invention to provide a wireless ring-type physical detector, which has a compact size that is easy to carry and may be carried for a long time without any uncomfortable feeling.

It is a further objective of the present invention to provide a wireless ring-type physical detector, which can help a doctor predict internal's problem accurately for making a prevention medication.

It is still a further objective of the present invention to provide a wireless ring-type physical detector, which can measure the heartbeat and the blood oxygen saturation and works as a pedometer.

### BRIEF DESCRIPTION OF THE DRAWINGS

FIG. 1 is a perspective view of a first preferred embodiment of the present invention;

FIG. 2 is a schematic view showing a display screen of the first preferred embodiment of the present invention;

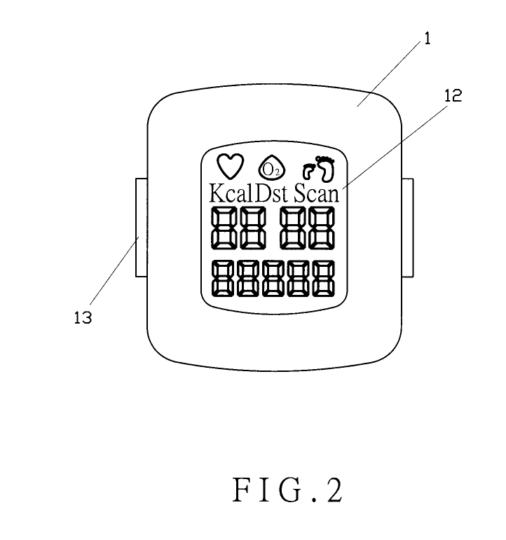

FIG. 3 is a schematic view of the first preferred embodiment of the present invention in use;

FIG. 4 is a schematic diagram of the first preferred embodiment of the present invention;

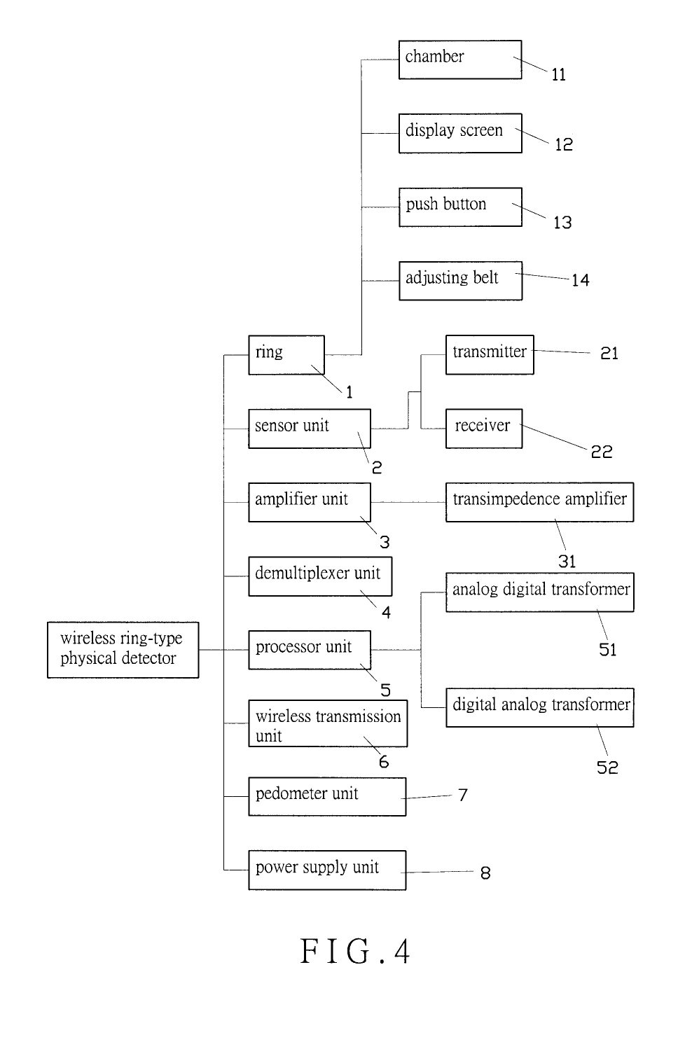

FIG. 5 is another schematic diagram of the first preferred embodiment of the present invention; and

FIG. 6 is a perspective view of a second preferred embodiment of the present invention.

### DETAILED DESCRIPTION OF THE PREFERRED EMBODIMENTS

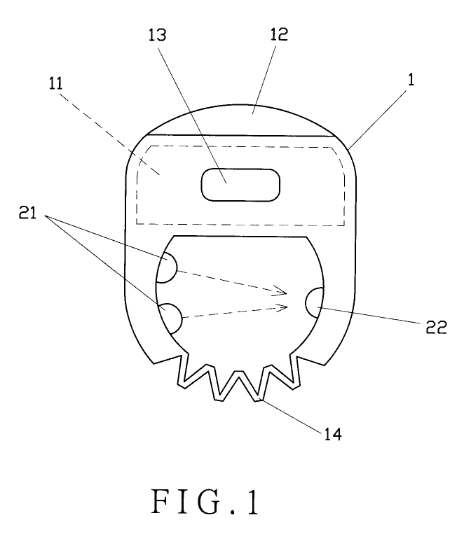

A wireless ring-type physical detector of the present invention is adapted for wearing on a user's finger, wrist, arm, neck or a waist. In the embodiments, a ring for a finger will be used. As shown in FIG. 1, a first preferred embodiment of the present invention comprises a ring 1, a sensor unit 2, an amplifier unit 3, a demultiplexer unit 4, a processor unit 5, a wireless transmission unit 6, a pedometer unit 7, and a power supply unit 8.

The ring 1 comprises a chamber 11, an inner surface, a display screen 12, a push button 13 for selecting an operating mode, and an adjusting belt 14 for changing the inner diameter of the ring 1.

The sensor unit 2 comprises two transmitters 21 and a receiver 22 which are in a light sensitive type and are disposed on the inner surface of the ring 1. The transmitters 21 and the receiver 22 are used to detect the size and the density of blood vessels and the thickness of blood for generating a light signal which is then transformed into a current signal. The two transmitters 21 are disposed on a first side of the inner surface of the ring 1, and the light signal from the transmitters 2 point at a first position at a second side of the inner surface of the ring 1. The receiver 22 is located at the first position.

The amplifier unit 3 is disposed in the chamber 11 of the ring 1. The sensor unit 2 may use a transimpedence amplifier (TIA) or a limiting amplifier for proceeding with the current signal from the sensor unit 2. In this embodiment, the sensor unit 2 uses a transimpedence amplifier 31 which is connected to the sensor unit 2. The transimpedence amplifier 31 transforms the current signal into a voltage signal.

The demultiplexer unit 4 is disposed in the chamber 11 of the ring 1 and connected to the amplifier unit 3. The demultiplexer unit 4 receives the voltage signal and outputs a demultiplex signal after selection and allotment.

The processor unit 5 is disposed in the chamber 11 of the ring 1 and connected to each unit and the push button 13 of the ring 1. The processor unit 5 is adapted to control each unit and receive the demultiplex signal for calculating to get a physical parameter. The physical parameter is one of the blood oxygen saturation, the heartbeat and the continuous blood pressure. The processor unit 5 comprises an analog digital transformer 51 and a digital analog transformer 52. The analog digital transformer 51 transforms the signal into a digital signal before it is transmitted to the processor unit 5, while the digital analog transformer 52 transforms a control signal from the processor unit 5 into an analog signal before transmission.

The wireless transmission unit 6 is disposed in the chamber 11 of the ring 1 and connected to the processor unit 5. Upon receiving the signal (physical parameter), the wireless transmission unit 6 will transmit the signal to a computer for storage or for analysis. The wireless transmission unit 6 is a wireless transmission module selected from one of infrared ray, Bluetooth, 5 KHz, 2.4 GHz, and Zigbee. In this embodiment, the wireless transmission unit 6 is a Bluetooth transmission module.

The pedometer unit 7 is disposed in the chamber 11 of the ring 1 and connected to the processor unit 5 for detecting the step vibration of the user.

The power supply unit 8 is disposed in the chamber 11 of the ring 1 and electrically connected to each of the aforesaid units for supplying power to each unit.

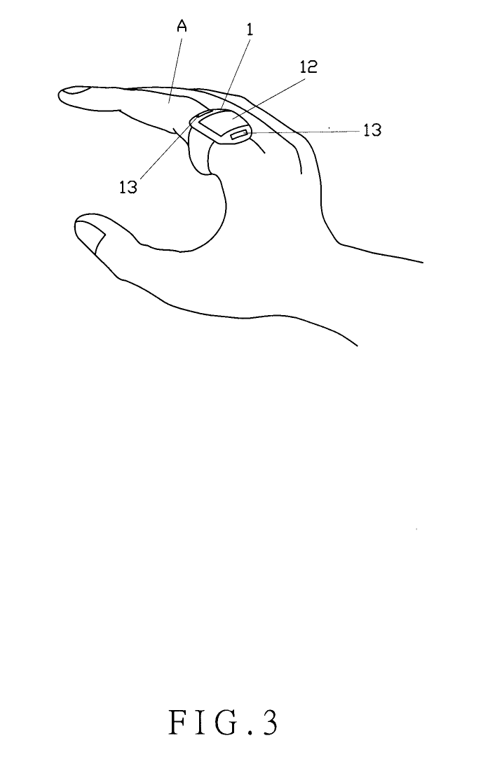

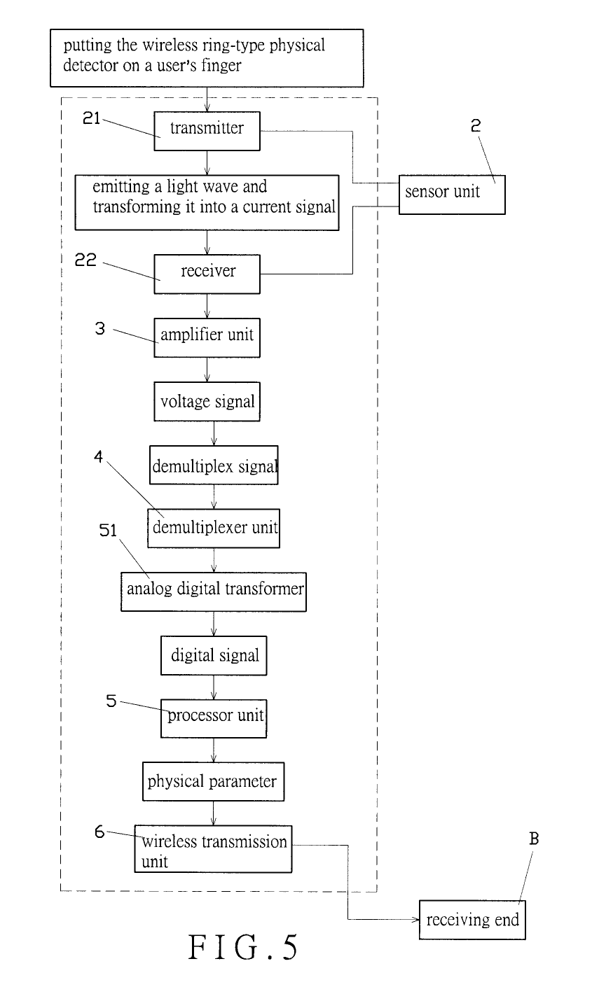

To operate the present invention, as shown in FIGS. 3 and 5, the wireless ring-type physical detector is put on a user's finger A and is adjusted by the adjusting belt 14 to the most appropriate position. The push button 13 on the ring 1 chooses the operating mode, such as the blood oxygen saturation, the heartbeat, the blood pressure, the pedometer mode, and so on. The two transmitters 21 and the receiver 22 of the sensor unit 2 are contact with the user's finger. The two transmitters 21 emit a light wave having a specific wavelength through the finger A to be received by the receiver 22. During transmission, the frequency and the wavelength of the light wave vary when it encounters with the blood vessel. This light wave will be transformed into a current signal and sent to the transimpedence amplifier 31 of the amplifier unit 3 for transforming the current signal into a voltage signal. The voltage signal is transmitted to the demultiplexer unit 4 for selection and allotment. The demultiplexer unit 4 outputs a demultiplex signal to the analog digital transformer 51 for transforming the demultiplex signal into a digital signal. The digital signal is transmitted to the processor unit 5 for calculating a physical parameter. The physical parameter is then transformed by the digital analog transformer 52 into an analog signal. The analog signal is transmitted through the wireless transmission unit 6 to an external receiving end B for storage or for analysis. The receiving end B may be a computer or any other electronic product, such as a PDA or a cell phone.

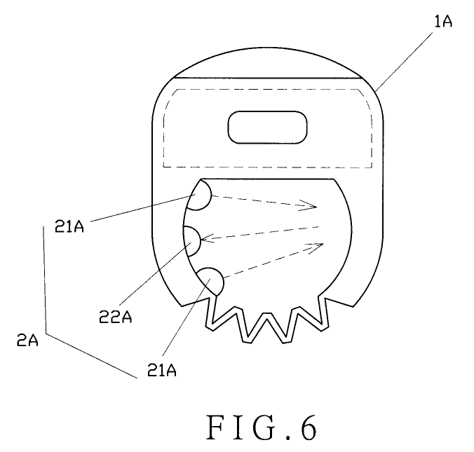

FIG. 6 shows a second preferred embodiment of the present invention, which is substantially similar to the first preferred embodiment with the exception described hereinafter. The transmitters 21A and the receiver 22A of the sensor unit 2A are disposed on the same side of the inner surface of the ring 1A. The light signals from the transmitters 21A point at the other side of the inner surface of the ring 1A and then reflect back to a second position where the receiver 22A is located. This design also provides a light induction effect.

The present invention utilizes the light signal to detect the size and the density of the blood vessel and the thickness of the blood. The information will be calculated to understand the continuous blood pressure. In the event that a high blood pressure is suspected, the present invention will assist the user to control the blood pressure at a normal status.

What is claimed is:

1. A wireless ring-type physical detector, comprising:
a ring, the ring comprising a chamber, an inner surface, and a display screen;
a sensor unit, the sensor unit comprising two transmitters and a receiver disposed on the inner surface of the ring, the transmitters and the receiver using a light signal to detect the blood oxygen saturation, the heartbeat and continuous blood pressure and transforming the light signal into a current signal;
an amplifier unit, the amplifier unit being disposed in the chamber of the ring and connected to the sensor unit for transforming the current signal into a voltage signal;
a demultiplexer unit, the demultiplexer unit being disposed in the chamber of the ring and connected to the amplifier unit for outputting a demultiplex signal after selection and allotment;
a processor unit, the processor unit being disposed in the chamber of the ring and connected with the sensor unit, the amplifier unit, and the demultiplexer unit for controlling each unit, the processor unit receiving and calculating the demultiplex signal to come up with a physical parameter and outputting the physical parameter to the display screen; and
a wireless transmission unit, the wireless transmission unit being disposed in the chamber of the ring and connected to the processor unit, the wireless transmission unit receiving and transmitting the physical parameter.

2. The wireless ring-type physical detector as claimed in claim 1, wherein the ring comprises a power supply unit therein.

3. The wireless ring-type physical detector as claimed in claim 1, wherein the two transmitters of the sensor unit are disposed on a first side of the inner surface of the ring, the light signal from the transmitters pointing at a first position at a second side of the inner surface of the ring, the receiver being located at the first position.

4. The wireless ring-type physical detector as claimed in claim 1, wherein the two transmitters of the sensor unit are disposed on a first side of the inner surface of the ring, the light signal from the transmitters pointing at a second side of the inner surface of the ring and returning back to a second position at the first side of the inner surface, the receiver being located at the second position.

5. The wireless ring-type physical detector as claimed in claim 1, wherein the amplifier unit is a transimpedence amplifier.

6. The wireless ring-type physical detector as claimed in claim 1, wherein the processor unit comprises an analog digital transformer for transforming a signal to be transmitted to the processor unit into a digital signal.

7. The wireless ring-type physical detector as claimed in claim 1, wherein the processor unit comprises a digital analog transformer for transforming an output signal into an analog signal before transmission.

8. The wireless ring-type physical detector as claimed in claim 1, wherein the wireless transmission unit is a wireless transmission module selected from one of infrared ray, Bluetooth, 5 KHz, 2.4 GHz, Zigbee.

9. The wireless ring-type physical detector as claimed in claim 1, wherein the physical parameter is one of the blood oxygen saturation, the heartbeat and the continuous blood pressure.

10. The wireless ring-type physical detector as claimed in claim 1, wherein the ring further comprises a push button, the push button being connected with the processor unit for selecting an operating mode.

11. The wireless ring-type physical detector as claimed in claim 1, wherein the ring further comprises an adjusting belt for changing an inner diameter of the ring.

12. The wireless ring-type physical detector as claimed in claim 1, further comprising a pedometer unit, the pedometer unit being disposed in the chamber of the ring and connected to the processor unit for sensing the step vibration of a user.
